(19) United States
(12) Patent Application Publication
(10) Pub. No.: US 2020/0055019 A1
(43) Pub. Date: Feb. 20, 2020

(54) ZEOLITE-CONTAINING ADSORBENT FOR SELECTIVE SEPARATION OF ISOMERS FROM AROMATIC HYDROCARBON MIXTURES, AND PRODUCTION AND USE OF SAME

(71) Applicant: CLARIANT INTERNATIONAL LTD, Muttenz (CH)

(72) Inventor: Stephan J. REITMEIER, Kirchseeon (DE)

(21) Appl. No.: 16/609,808

(22) PCT Filed: May 4, 2018
(86) PCT No.: PCT/EP2018/061464

§ 371 Date: Oct. 31, 2019

(30) Foreign Application Priority Data
May 9, 2017 (DE) 10 2017 207 817.0

(51) Int. Cl. B01J 20/18; B01D 53/02; B01D 15/08; B01J 20/28

(52) U.S. Cl. CPC B01J 20/186; B01D 53/02; B01D 2257/7027; B01J 20/28019; B01D 2253/1085; B01D 15/08

(57) ABSTRACT

The present invention relates to an improved adsorbent for separating para-xylene from a mixture of xylene isomers, characterized in that the adsorbent contains a barium-containing zeolite of the faujasite type whose exchangeable sites are occupied to an extent of at least 4.0% by magnesium ions. The present invention additionally relates to the production of the adsorbent and to the use thereof in the separation of para-xylene from a mixture of xylene isomers.

The present invention relates to an adsorbent based on a barium- and magnesium-exchanged zeolite of the faujasite type which displays, in particular, an increased separation action in respect of xylene isomers. The invention additionally relates to the production of the adsorbent and also the use thereof in the selective separation of isomers from aromatic hydrocarbon mixtures.

Aromatic hydrocarbons such as xylenes are important raw materials of the chemical industry and are used, in particular, as solvents or for producing plastics. Here, for example, para-xylene is used for preparing terephthalic acid, ortho-xylene serves for obtaining phthalic acid or phthalic anhydride.

Owing to their similar boiling points, separation of the C8-aromatic isomers para-xylene, ortho-xylene and meta-xylene and also ethylbenzene by distillation is difficult. One method employed in the industry is to fractionate the isomer mixture adsorptively by means of an adsorbent having a suitable separation action.

Materials based on barium- and/or potassium-exchanged zeolites of the faujasite type, FAU, have been found to be particularly suitable adsorbents. With regard to the nomenclature of the abovementioned topology, reference is made to the “Atlas of Zeolite Framework Types”, Ch. Baerlocher, 6th Edition, 2007, which gives an overview of the various topologies of the zeolite structures and the disclosure of which is in this respect incorporated into the description. Zeolites of the zeolite X type have been found to be preferred representatives of the zeolites having FAU topology.

U.S. Pat. No. 8,530,367 B2 describes a process for the selective separation of para-xylene from a xylene isomer mixture. The adsorbent used is based on zeolite X whose exchangeable cationic sites are occupied to an extent of at least 90% either by barium ions or barium ions and potassium ions, with the number of the sites occupied by potassium ions making up to ⅓ of the sites occupied by barium and potassium ions. Any sites not occupied by barium and/or potassium ions are occupied by alkali metal ions or alkaline earth metal ions other than barium ions.

WO 2012/134973 A2 describes a process for producing zeolite X-containing adsorbents by conversion of binder material into zeolite X in order to increase the proportion of zeolite X in the adsorbent and to effect an increase in active composition. The adsorbent is obtained by barium and potassium ion exchanges.

The adsorbent described in U.S. Pat. No. 8,791,039 B2 comprises, inter alia, a zeolite X whose exchangeable sites are occupied by group IIA metals and/or potassium. The adsorbent displays, according to the description, a higher adsorption capacity and a more rapid mass transfer rate.

WO 2014/090771 A1 describes an adsorbent comprising zeolite X crystals having an average diameter of ≤1.7 μm and a nonzeolitic binder component which displays an increased adsorption capacity and mechanical strength. Alkaline metal and alkaline earth metal oxides other than barium oxide and potassium oxide are preferably present in a proportion by weight of less than 5% based on the total mass of the adsorbent; a technical effect of these alkali metal and alkaline earth metal oxides is not described.

There continues to be a need for improved adsorbents which have an increased separation action in respect of aromatic hydrocarbon isomers, in particular in respect of the separation of para-xylene from C8-aromatic isomer mixtures.

It was therefore an object of the present invention to provide an adsorbent which displays an increased separation factor for para-xylene in respect of ortho-xylene and meta-xylene combined with a high adsorption capacity.

This object is achieved by the zeolite-containing adsorbent of the invention which is characterized in that at least 4.0% of the exchangeable sites of the barium-containing faujasite are occupied by magnesium ions.

The invention provides an adsorbent for separating aromatic hydrocarbon isomers, in particular xylene isomers, comprising a support material containing a barium-containing zeolite of the faujasite type, preferably of the zeolite X type, characterized in that at least 4.0% of the exchangeable sites of the barium-containing zeolite in the adsorbent are occupied by magnesium ions, and also the production thereof and the use thereof as adsorbent in the separation of aromatic hydrocarbon isomers, in particular xylene isomers.

The adsorbent of the invention is produced by means of the following steps according to the invention:
a) provision of a support material comprising a zeolite of the faujasite type,
b) ion exchange of the support material from step a) with barium ions and magnesium ions in an aqueous medium,
c) drying of the support material obtained after step b) to give an adsorbent,
   wherein at least 4.0% of the exchangeable sites of the barium-containing zeolite in the adsorbent obtained after step c) are occupied by magnesium ions.

The ion exchanges with barium ions and magnesium ions are carried out by introducing the amount of ions into the support material either in a single exchange step or in a plurality of successive steps. The at least one exchange step can be carried out in each case at room temperature or elevated temperature of up to 100° C. Drying of the support material or of the partially ion-exchanged support material can optionally be carried out between the individual ion exchanges. The drying temperature here has to be selected in such a way that no damage to the zeolite structure occurs. The temperature is preferably up to 80° C., more preferably up to 60° C.

In a preferred embodiment, the support material is, in step b), firstly subjected to barium ion exchange and subsequently to magnesium ion exchange. The barium ion exchange can be carried out either in a single step or in the form of a plurality of successive steps. The barium ion exchange is followed by the magnesium ion exchange. This can likewise be carried out in a single step or in a plurality of successive steps. The support material can be dried, preferably below a temperature of 80° C., more preferably up to 60° C., between the exchange steps.

In another preferred embodiment, the support material is, in step b), firstly subjected to magnesium ion exchange and subsequently to barium ion exchange. The magnesium ion exchange can be carried out either in a single step or in the form of a plurality of successive steps. The magnesium ion exchange is followed by the barium ion exchange. This can likewise be carried out in a single step or in a plurality of successive steps. The support material can be dried, preferably below a temperature of 80° C., more preferably up to 60° C., between the exchange steps.

Suitable starting compounds for the exchange with barium ions are in principle all compounds which are soluble in water, basic or acidic aqueous solutions. Preference is given to using barium nitrates or halides.

Suitable starting compounds for the exchange with magnesium ions are in principle all compounds which are soluble in water, basic or acidic aqueous solutions. Preference is here given to magnesium nitrates, sulfates or halides, more preferably magnesium nitrates or halides.

For the purposes of the present invention, an aqueous medium is an aqueous solution in which the Ba or Mg compounds are present in dissolved form. The solution can contain not only the appropriate Ba or Mg compounds but also further constituents such as acids or bases in order to dissolve the Ba or Mg compounds completely.

The material obtained after step b) is subjected in step c) to drying in order to obtain an adsorbent. This drying preferably takes place at a temperature of up to 80° C., more preferably up to 60° C. The duration is preferably from 0.5 h to 24 h, more preferably from 2.0 h to 20 h.

In a preferred embodiment, the adsorbent obtained after step c) is additionally subjected to activation in a step d) in order to set the water content of the adsorbent to such a value that the adsorption properties are optimized. The activation is carried out at a temperature in the range from 100° C. to 450° C., preferably in the range from 100° C. to 300° C. The duration is in the range from 1.0 h to 16 h, preferably from 1.0 h to 8.0 h.

The support material comprises a zeolite of the faujasite type. This zeolite has an atomic Si/Al ratio in the range from 1.00 to 3.00. The zeolite of the faujasite type is preferably a zeolite X having an Si/Al ratio of from 1.15 to 1.50, more preferably an Si/Al ratio of from 1.15 to 1.25.

The support material can be present in various forms. Illustrative embodiments are granules, spheres, extrudates or pellets or other shaped bodies, with the support material preferably being present in the form of granules or spheres, most preferably as spheres. For shaping the spheres, it is possible to use various production methods, e.g. agglomeration or granulation processes, for example in a pelletizing pan or Eirich mixer, spray drying processes, extrusion with subsequent grounding or oil drop processes.

When the support material is present in the form of spheres, these typically have particle size distributions with average diameters of less than 1.5 mm, preferably less than 1.0 mm, more preferably in the range from 0.3 mm to 1.5 mm, even more preferably from 0.3 mm to 1.0 mm and most preferably in the range from 0.4 mm to 0.8 mm.

In one embodiment, the support material comprises a binder. Suitable binders are the compounds known from the prior art, e.g. alumina, silica, silica-alumina, ceramics, clay minerals such as bentonite, kaolin, kaolinite, metakaolin, nacrite, halloysite, dickite, attapulgite, sepiolite, montmorillonite, illite or other oxide-containing compounds. The proportion of binder in the adsorbent is preferably from 5.0 to 30% by weight, more preferably from 5.0 to 20% by weight. The presence of the binder brings about, for example, an increase in the strength of the support material, which is an important factor especially when the support material is used in industrial plants for the selective adsorption of aromatic compounds.

The use of binders, in particular naturally occurring binders such as kaolin, kaolinite, metakaolin or bentonite, usually results in a certain amount of magnesium being present in the support material before step b). However, this proportion is so small that it does not exert any significant influence on the adsorption properties. The proportion of magnesium in the binder used is usually less than 0.20% by weight, preferably less than 0.15% by weight.

In a further embodiment, the adsorbent comprises a binder-free support material. This is, for the purposes of the present invention, a material in which the zeolite particles are bound to one another essentially by means of other zeolite particles. In contrast to a binder-containing support material, the binder-free support material consists entirely of the zeolite required for the adsorption function and nevertheless has a satisfactory strength as a result of the bonding of the zeolite particles among one another. These other zeolite particles are obtained by transformation of suitable clay minerals such as kaolin, kaolinite or metakaolin by means of zeolitization in a hydrothermal reaction. Here, the support material containing zeolite and clay mineral and optionally additives such as a silicon source is thermally treated in an alkaline solution, with the aqueous alkaline solution optionally also containing one or more silicon sources and/or aluminium sources which are required for converting the clay mineral into a faujasite structure having an atomic Si/Al ratio in the required range. Otherwise known process control thus enables the proportion of the nonzeolitic binder based on a clay mineral to be converted into faujasite particles which form bonds directly to the existing faujasite particles and lead to a material which displays, in particular, a high strength. The clay minerals used in this process can, as natural raw materials, comprise, in particular, a certain amount of magnesium in the form of impurities. However, this proportion is so small that it does not exert any significant influence on the adsorption properties. The proportion of magnesium in binders typically used is usually less than 0.20% by weight, preferably 0.15% by weight or less.

In a preferred embodiment, the support material does not contain any zeolites other than those of the faujasite type, preferably of the zeolite X type.

In a particularly preferred embodiment, the support material is binder-free and consists only of zeolite of the faujasite type, preferably of the zeolite X type.

The adsorbent of the invention has, after the magnesium ion exchange, such a proportion of Mg that at least 4.0%, preferably at least 7.0%, most preferably at least 10.0%, of the exchangeable sites of the barium-containing faujasite in the adsorbent are occupied by magnesium ions, preferably from 4.0% to 20.0% of the exchangeable sites of the barium-containing faujasite in the adsorbent are occupied by magnesium ions, more preferably from 10.0% to 20.0% of the exchangeable sites of the barium-containing faujasite in the adsorbent are occupied by magnesium ions.

If part of the silicon atoms in a zeolite structure has been replaced by aluminium atoms, this results in a negative framework charge which is compensated for by cations in the voids of the zeolite structure. The number of exchangeable sites for the purposes of the present invention then corresponds to the sum of all cations present, in each case multiplied by the valency thereof:

Number of exchangeable sites=Σⱼ(cation(j)*valency(j))

The percentage of the exchangeable sites occupied by magnesium ions is the ratio of the total number of all magnesium ions multiplied by the valency thereof to the number of exchangeable sites.

The proportion of barium in the adsorbent of the invention is not more than 36.0% by weight, preferably in the range from 25.0% by weight to 36.0% by weight, more preferably in the range from 26.0% by weight to 36.0% by weight, particularly preferably in the range from 28.0% by weight to 34.0% by weight, based on the mass of the adsorbent.

Apart from magnesium and barium, the adsorbent of the invention can additionally comprise alkali metal elements such as sodium or potassium. The proportion of these is preferably less than 5.0% by weight, more preferably less than 2.0% by weight and particularly preferably less than 0.5% by weight, based on the mass of the adsorbent.

The lateral compressive strength per diameter (LCSD) of the adsorbent of the invention is greater than or equal to 2.0 N/mm, preferably greater than or equal to 3.0 N/mm. It is preferably in the range from 2.0 N/mm to 10.0 N/mm, preferably from 3.0 N/mm to 7.0 N/mm, most preferably from 4.0 N/mm to 7.0 N/mm.

The BET surface area of the adsorbent of the invention is in the range from 500 to 800 m²/g, preferably in the range from 550 to 750 m²/g and particularly preferably in the range from 600 to 700 m²/g.

The compressive strength (also known as bulk crush strength, BCS) of the adsorbent of the invention is greater than or equal to 1.5 MPa, preferably greater than or equal to 2.0 MPa, most preferably greater than or equal to 3.0 MPa. It is typically in the range from 1.5 MPa to 4.0 MPa, preferably in the range from 2.0M Pa to 4.0 MPa, most preferably in the range from 3.0 MPa to 4.0 MPa.

The adsorbent of the invention is suitable for a process for separating aromatic hydrocarbons from a mixture of the corresponding isomers. It is particularly suitable for separating para-xylene from a mixture containing at least one further isomer from among the other C8-aromatic isomers, e.g. ortho-xylene or meta-xylene. Typical mixtures contain para-xylene together with the isomers ortho- and meta-xylene and optionally ethylbenzene in various weight distributions.

This process can be employed both for a liquid isomer mixture and for a gaseous stream consisting of the C8-aromatic isomers. The adsorption process can be carried out by means of moving-bed or fixed-bed technology, continuously or batchwise, preferably by means of simulated moving bed processes in concurrent or countercurrent (also known as “cocurrent or countercurrent simulated moving bed”).

In the process of adsorptive separation, the adsorbent of the invention is brought into contact with the appropriate mixture containing the isomers to be separated under conditions under which selective adsorption of the desired isomers takes place. The adsorption temperatures here are in the range from 100° C. to 200° C., preferably in the range from 150° C. to 180° C., and the adsorption process proceeds in the pressure range from slightly above atmospheric pressure to about 3.5 MPa. It typically occurs at pressures between 0.7 MPa and 2.0 MPa.

Carrying out the adsorptive, chromatographic separation of, in particular, the C8-aromatic isomers in the liquid phase is of particular interest. Here, a stream consisting of the C8-aromatic isomers is usually brought into contact with the adsorbent, with the adsorption temperature being in a range from 100° C. to 200° C., preferably from 150° C. to 180° C. The pressure is between 0.11 MPa and 3.5 MPa.

Before use for adsorption of C8-aromatic isomers, the adsorbent is usually subjected to an activation. This is known from the prior art and is carried out in order to set the water content of the adsorbent in such a way that the separation action and the adsorption capability are optimized. Here, the adsorbent is subjected to a thermal treatment. The activation is carried out at a temperature in the range from 100° C. to 450° C., preferably in the range from 100° C. to 300° C. The duration of the activation is selected so that the water content is reduced to the desired value, and is typically between 1.0 h and 16 h, preferably between 1.0 h and 8.0 h.

During the contacting of the adsorbent with the mixture in the form of a continuous or batch process, para-xylene is preferentially adsorbed into the pores of the adsorbent compared to the other C8-aromatic isomers in the mixture; under these conditions, a preferential separation of the para-xylene takes place. As a result, the adsorbed phase (which is present in the pores of the zeolite) is selectively enriched with para-xylene, relative to the other components of the mixture (i.e. the other C8-aromatic isomers). The remaining mixture which has been depleted in para-xylene represents the unadsorbed phase. If, for example, the mixture comprises the isomers ortho- and meta-xylene in addition to para-xylene, the adsorbed phase has a selectively increased proportion of para-xylene and the remaining, unadsorbed phase is enriched in ortho- and meta-xylene, in each case relative to the starting mixture. The unadsorbed phase can be removed as raffinate mixture from the adsorbent by means of a desorbent or eluent. The adsorbed phase which has been enriched in para-xylene is separately flushed, i.e. desorbed, as extract mixture from the adsorbent by means of a desorbent or eluent.

Possible desorbents or eluents are, in particular, aromatic hydrocarbons, for example para-diethylbenzene, para-diisopropylbenzene, toluene or other 1,4-substituted benzenes, and also mixtures thereof. However, the components mentioned represent only a selection and should not be regarded as exhaustive by a person skilled in the art. To effect desorption, the desorbent is passed over the bed of adsorbent material. The para-xylene with which the extract stream is enriched and also the other C8-aromatic isomers present in the raffinate stream are subsequently separated from the desorbent, e.g. by distillation. Here, para-xylene having a high purity is obtained. The selectivity in respect of para-xylene is particularly critical to the optimal separation action of the adsorbent used.

The selectivity of the adsorbent in respect of the capability of separating off para-xylene is characterized by the separation factor or the selectivity/3. This is defined according to the following equation (1):

$$β ( p X / i ) = \frac{( \frac{{pX}_{A}}{{i}_{A}} )}{( \frac{{pX}_{L}}{{i}_{L}} )} \qquad ( 1 )$$

Here, pX_{A} is the amount of para-xylene adsorbed at equilibrium by the adsorbent, i_{A} is the amount of the other C8-aromatic isomer (e.g. oX_{A} for ortho-xylene) adsorbed by the adsorbent, pX_{L} is the amount of para-xylene present at equilibrium in the solution and i_{L} is the amount of the other C8-aromatic isomer (e.g. oX_{L} for ortho-xylene) present in the solution. The amount of para-xylene adsorbed at equilibrium by the adsorbent and of the other C8-aromatic isomer (e.g. ortho-xylene) can be calculated from the mass balance of the initial composition of the solution and the composition of the solution present at equilibrium. For this purpose, it is possible, for example, to determine in each case the concentration by weight of the xylenes present in the initial solution mixture and the mixture after attainment of equilibrium by gas-chromatographic analysis.

In order to be able to be used industrially as adsorbent in the separation of para-xylene, the adsorbent has to have not only an improved selective separation of para-xylene but also a sufficiently large adsorption capacity. This ensures that even small amounts of adsorbent can adsorb enough para-xylene in order to achieve the desired purities and productivity, and the required amount of adsorbent can correspondingly be reduced.

Industrially utilized adsorbents based on zeolites having a faujasite topology and a suitable adsorption capacity typically have micropore volumes, determined by means of nitrogen adsorption and t-plot evaluation, in the range from 0.24 to 0.29 ml/g.

The weight-based adsorption capacity of the adsorbent C_{ads}, determined from, for example, batch tests on the equilibrium adsorption, can be employed as measure of the available adsorption capacity of the adsorbent. The weight-based adsorption capacity, reported in % by weight, is calculated from the sum of the masses of all species adsorbed at equilibrium, based on the mass of the adsorbent used. In the case of the adsorbent of the invention, the adsorption capacity C_{ads} is between 13.0% and 18.0% g/(g of adsorbent), preferably between 14.0% and 17.0% g/(g of adsorbent), most preferably greater than 15.0% g/(g of adsorbent).

### EXAMPLES

The determination of the lateral compressive strength was carried out using a Zwick Z 0.5 instrument from Zwick/Roell GmbH. Here a support was positioned and a punch located vertically above this was moved in the direction of the support until it reached the sphere and an increase in force of 0.1 N was detected. This point defined the height of the sphere, based on the height of the support. The punch was subsequently moved further, with an increase in force of 1 N/s being set. The absolute increase in force was measured and the procedure was continued until a decrease in the force by 30% was measured. The maximum value of the applied force could be determined in this way. This measurement was carried out on at least 50 individual spheres and the arithmetic mean lateral compressive strength was calculated.

The measurement of the compressive strength (also known as bulk crush strength, BCS) was carried out in accordance with SMS1471 (Shell Method Series SMS 1471-74, “Determination of Bulk Crushing Strength of Catalysts. Compression-Sieve method”). A Zwick Roell Z020 compressive strength measuring instrument from Zwick/Roell GmbH was used for the measurement.

To separate off fines formed in the measurement, a sieve having a mesh opening of 200 μm was used, which constitutes a departure from SMS1471-74.

A sample having a volume of about 20 cm³ was sieved by means of the sieve to separate off adhering fines and dried at 250° C. for at least 2 hours in a drying oven. This sample was subsequently introduced into a cylinder having a clearly defined diameter. To achieve better pressure distribution, a bed of stainless steel balls was placed on top of the bed of the adsorbent. A pressure was then exerted on the sample by means of a punch, and this pressure was continuously increased. After the end of the measurement, the stainless steel balls were separated off and the bed of the adsorbent was transferred to the analytical sieve; fines remaining in the cylinder were transferred into the sieve with the aid of a brush.

Removal of the fragments and fines adhering to the shaped adsorbent bodies was subsequently carried out by means of sieving. The shaped adsorbent bodies were subsequently transferred back into the cylinder and the measurement with increasing pressure values exerted on the sample was repeated until the cumulated proportion of fines attained a value of 0.5% by weight. The accuracy of the measurement method is ±0.1 MPa.

The BET surface areas were determined in accordance with DIN 66135. To determine the micropore volume and the micropore surface area, the adsorption isotherm of nitrogen at the temperature of liquid nitrogen (77 K) was determined using the ASAP 2020 M from Micromeritics.

The sample was firstly baked under reduced pressure (<5 μm of Hg) in the sample tube. The nitrogen adsorption isotherm was recorded in the relative pressure range p/p0 from 0.001 to 1, with at least 35 measurement points being determined.

The micropore volume and the micropore surface area was evaluated and determined from the nitrogen adsorption isotherm by means of the t-plot method: in the t-plot graph, the adsorbed volume was plotted as a function of the multilayer thickness (t) according to the equation of Harkins-Jura. The points of the t-plot curve which lay on a straight line were defined, usually in the range from 0.35 nm to 0.65 nm. The micropore volume is obtained from the Y-axis intersect of the straight lines.

To determine the pore volume and the pore distribution, the PASCAL 440 mercury porosimeter from Thermo Electron Corporation was used. The measurement was carried out in accordance with ASTM-D4284-12.

The sample was evacuated (p<0.01 mbar) at room temperature for 30 minutes in a dilatometer and filled with mercury. After placing the PASCAL 440 in an autoclave, the pressure was slowly increased to 4000 bar gauge. The evaluation was carried out assuming cylindrical pores, a contact angle of 140° and a surface tension of mercury of 480 dyn/cm. The pore distribution was obtained in the pore radius range from 7500 nm to 1.8 nm (in the case of measurement up to 4000 bar gauge) or up to 3.6 nm (in the case of measurement up to 2000 bar gauge).

Determination of the proportion of the zeolite material in the adsorbent is carried out by means of the method of X-ray powder diffraction which is known to those skilled in the art. A D4 Endeavor from BRUKER and CuKα1 radiation (wavelength 1.54060 Å, 40 kV, 35 mA) is used for this purpose. The sample is measured over a range from 5 to 90° 28 (in steps of 0.020° 2, 1.5 seconds measurement time per step). The proportion of the zeolite material in the sample is determined from the resulting diffraction pattern using the TOPAS software from BRUKER.

The determination of the chemical composition was carried out by means of elemental analyses using X-ray fluorescence analysis in accordance with DIN 51001 and on the basis of the method of DIN EN ISO 12677. The samples were for this purpose finely milled in order to achieve homogeneous distribution of the particles in the sample and dried at 105° C. This material was mixed with Li₂B₄O₇ and pressed to give a pellet.

Relative proportion by weight always relates, for the purposes of the present patent application, to samples after loss on ignition.

The loss on ignition was determined by heating a sample of the adsorbent to 1000° C. under an air atmosphere in a muffle furnace and keeping it at this temperature for 3.0 h. The loss on ignition is calculated as the difference between the mass of the weighed-in sample before the thermal treatment and the residual mass after the thermal treatment. Apart from carrying out the loss on ignition determination in a muffle furnace, other methods such as Karl Fischer determination (ASTM D1364) can also be employed, as long as these give analogous results on comparing the methods.

### Comparative Example 1

As starting material, use was made of spherical binder-free shaped adsorbent bodies which had an average diameter of 0.7 mm, consisted essentially of zeolite NaX having an atomic Si/Al ratio of 1.17 and had been prepared by granulation using the production methods known from the prior art.

For the ion exchange, 40 kg of an 8.7% strength by weight solution of BaCl₂.2 H₂O in deionized H₂O was prepared and this was heated to 80° C. 2000 g of the shaped adsorbent bodies, based on the dry mass, were introduced into this solution and the mixture was stirred at 80° C. for 2 h. The salt solution was subsequently separated off at this temperature and the treated adsorbent was used again for the next ion exchange. For this purpose, 40 kg of a 17.4% strength by weight solution of BaCl₂.2 H₂O in deionized H₂O was prepared and heated to 80° C., the previously treated adsorbent was introduced into this solution and the mixture was stirred at 80° C. for 2 h. The salt solution was subsequently separated off immediately and the remaining adsorbent was divided into two parts having the same weight.

One part was washed with deionized H₂O until the conductivity of the washing water was below 100 μS/cm. The washed product obtained was then dried at 60° C. for 16 h in a convection drying oven. The product obtained served as starting material adsorbent A for Examples 2 and 3 according to the invention. The properties of the prepared product adsorbent A are summarized in Table 1.

### Comparative Example 2

The other part of the adsorbent from Comparative Example 1 which had neither been washed nor dried was subjected to a third barium exchange. For this purpose, 20 kg of a 17.4% strength by weight solution of BaCl₂.2 H₂O in deionized H₂O was prepared and heated to 80° C., the adsorbent which had previously been treated twice was introduced into this solution and the mixture was stirred at 80° C. for 2 h. The salt solution was subsequently separated off at this temperature and the remaining adsorbent was washed with deionized H₂O until the conductivity of the washing water was below 100 μS/cm. The product was then dried at 60° C. for 16 h in a convection drying oven.

The product obtained served as starting material adsorbent B for Examples 4, 5 and 6 according to the invention. The properties of the prepared product adsorbent B are summarized in Table 1.

### Example 1

100 g, based on the dry mass, of adsorbent A were introduced into 1053 g of a 5.0% strength by weight solution of MgCl₂.6 H₂O in deionized H₂O and this mixture was stirred at room temperature for 2 h. The solution was subsequently separated off and the remaining product was used without further drying for the next experiment.

### Example 2

The unwashed and undried part from Example 1 was introduced into 1053 g of a 5.0% strength by weight solution of MgCl₂.6 H₂O in deionized H₂O and this mixture was stirred at room temperature for 2 h. The solution was subsequently separated off and the remaining product was divided into two parts in a weight ratio of 1:1. The one half was washed with deionized H₂O until the conductivity was below 100 μS/cm. It was subsequently dried at 60° C. for 16 h. In the following, this product will be referred to as adsorbent 1. The properties of the prepared product adsorbent 1 are summarized in Table 1. The other part was used for the next experiment.

### Example 3

For the ion exchange, 530 g of a 5.0% strength by weight solution of MgCl₂.6 H₂O in deionized H₂O was prepared and this was heated to 60° C. The unwashed and undried part from Example 2 was introduced into this solution and this mixture was stirred at 60° C. for 2 h. The solution was subsequently separated off at this temperature and the remaining product was washed with deionized H₂O until the conductivity was below 100 μS/cm. It was subsequently dried at 60° C. for 16 h. In the following, this product will be referred to as adsorbent 2. The properties of the prepared product adsorbent 2 are summarized in Table 1.

### Example 4

150 g, based on the dry mass, of adsorbent B were introduced into 1579 g of a 5.0% strength by weight solution of MgCl₂.6 H₂O in deionized H₂O and this mixture was stirred at room temperature for 2 h. The solution was subsequently separated off and the remaining product was divided into two parts in a weight ratio of 1:2. One part, which corresponds to one third of the mass of the remaining product, was washed with deionized H₂O until the conductivity was below 100 μS/cm. It was subsequently dried at 60° C. for 16 h. In the following, this product will be referred to as adsorbent 3. The properties of the prepared product adsorbent 3 are summarized in Table 1. The other part was used for the next experiment.

### Example 5

The unwashed and undried part from Example 4 was introduced into 1053 g of a 5.0% strength by weight solution of MgCl₂.6 H₂O in deionized H₂O and this mixture was stirred at room temperature for 2 h. The solution was subsequently separated off and the remaining product was divided into two parts in a weight ratio of 1:1. The one half was washed with deionized H₂O until the conductivity was below 100 μS/cm. It was subsequently dried at 60° C. for 16 h. In the following, this product will be referred to as adsorbent 4. The properties of the prepared product adsorbent 4 are summarized in Table 4. The other part was used for the next experiment.

### Example 6

For the ion exchange, 530 g of a 5.0% strength by weight solution of MgCl₂.6 H₂O in deionized H₂O was prepared and this was heated to 60° C. The unwashed and undried part from Example 5 was introduced into this solution and this mixture was stirred at 60° C. for 2 h. The solution was subsequently separated off at this temperature and the remaining product was washed with deionized H₂O until the conductivity was below 100 μS/cm. It was subsequently dried at 60° C. for 16 h. In the following, this product will be referred to as adsorbent 5. The properties of the prepared product adsorbent 5 are summarized in Table 1.

TABLE 1
Properties of the prepared adsorbents

| | Proportion | Proportion | | | Lateral | | | | BET |
|---|---|---|---|---|---|---|---|---|---|
| | of Ba | of Mg | Degree of | Atomic | compressive | Compressive | Adsorption | Micropore | surface |
| | [% by | [% by | Mg exchange | Si/Al | strength LCSD | strength | capacity C_{ads} | volume | area |
| Adsorbent | weight] | weight] | [%] | ratio | [N/mm] | [MPa] | [g/g_{adsorbent}] | [mL/g] | [m²/g] |
| Adsorbent A | 33.05 | 0.03 | 0.0 | 1.16 | 8.23 | 3.6 | 14.7 | 0.258 | 662 |
| Adsorbent B | 34.12 | 0.03 | 0.0 | 1.16 | 7.09 | 3.4 | 15.6 | 0.255 | 650 |
| Adsorbent 1 | 30.54 | 0.47 | 7.6 | 1.16 | 6.13 | 3.5 | 15.6 | 0.259 | 664 |
| Adsorbent 2 | 29.74 | 0.82 | 13.0 | 1.17 | 6.00 | 3.1 | 16.4 | 0.263 | 675 |
| Adsorbent 3 | 32.24 | 0.27 | 4.4 | 1.17 | 8.00 | 3.4 | 15.1 | 0.254 | 651 |
| Adsorbent 4 | 31.44 | 0.43 | 7.2 | 1.17 | 5.97 | 3.0 | 15.6 | 0.260 | 666 |
| Adsorbent 5 | 30.27 | 0.75 | 12.1 | 1.16 | 6.57 | 3.3 | 15.9 | 0.262 | 672 |

### Use Example 1

The adsorbents produced in the experimental part were used in a process in order to determine the suitability for the selective separation of para-xylene (pX) from the isomers ortho-xylene (oX) and meta-xylene (mX). For this purpose, they were firstly thermally activated at 220° C. for 2 h and in each case 0.5 g of the thermally activated adsorbent was then transferred into a pressure vessel for a batch test to determine the equilibrium adsorption. 3.5 g of solution containing equal proportions by mass of 2.0% by weight of each of para-xylene, meta-xylene and ortho-xylene and also ethylbenzene and para-diethylbenzene in isooctane as solvent were introduced into this vessel. The pressure during the adsorption test was 9 bar gauge±0.5 bar gauge, and the adsorption temperature was 177° C. Under these conditions, the adsorbent was equilibrated in the vessel for 4 h until thermodynamic equilibrium had been established and during this time was stirred at intervals in order to equalize concentration differences within the solution.

The chemical composition of the solution was determined before and after the adsorption test in each case by means of gas-chromatographic analysis. The amount of the respective substance which had been adsorbed by the adsorbent could be calculated from the difference between the compositions. The separation factors β(pX/i) can thus be calculated according to equation (1). The results are shown in Table 2.

TABLE 2
Overview of separation factors β (pX/i)
for i = ortho-xylene and meta-xylene.

| Example | β (pX/oX) | β (pX/mX) |
|---|---|---|
| Adsorbent A | 3.22 | 3.19 |
| Adsorbent B | 3.19 | 3.14 |
| Adsorbent 1 | 3.23 | 3.19 |
| Adsorbent 2 | 3.30 | 3.25 |
| Adsorbent 3 | 3.33 | 3.24 |
| Adsorbent 4 | 3.42 | 3.32 |
| Adsorbent 5 | 3.50 | 3.39 |

It is clear from the data that an adsorbent which comprises an Mg-exchanged barium-containing zeolite of the faujasite-type and in which at least 4.0% of the exchangeable sites of the zeolite are occupied by Mg displays a significantly improved selectivity for para-xylene compared to an adsorbent produced according to the prior art. This trend is clear even in the case of an adsorbent having a comparatively low proportion of barium, and is even more evident when the data for adsorbent B are compared with those for adsorbents 3 to 5.

1. An Adsorbent for separating xylene isomers, comprising a support material containing a barium-containing zeolite of the faujasite type, characterized in that at least 4.0% of the exchangeable sites of the barium-containing faujasite are occupied by magnesium ions.

2. An Adsorbent according to claim 1, wherein the zeolite is a zeolite X.

3. An Adsorbent according to claim 1, wherein the support material is binder-free.

4. An Adsorbent according to claim 1, wherein the support material is present in spherical form.

5. An Adsorbent according to claim 1, wherein the Si/Al ratio of the zeolite is between 1.0 and 1.5, preferably between 1.15 and 1.25.

6. An Adsorbent according to claim 1, wherein at least 7.0%, preferably at least 10.0%, of the exchangeable sites are occupied by magnesium ions.

7. An Adsorbent according to claim 1, wherein the barium content is between 25.0% by weight and 36.0% by weight, based on the mass of the adsorbent.

8. A Process for producing an adsorbent for separating xylene isomers according to claim 1, comprising the following steps:
a) provision of a support material comprising a zeolite of the faujasite type,
b) ion exchange of the support material from step a) with barium ions and magnesium ions in an aqueous medium,
c) drying of the support material obtained after step b) to give an adsorbent,
wherein at least 4.0% of the exchangeable sites of the barium-containing zeolite in the adsorbent obtained after step c) are occupied by magnesium ions.

9. A Process according to claim 8, wherein at least 7.0%, preferably at least 10.0%, of the exchangeable sites are occupied by magnesium.

10. A Process according to claim 8, wherein the support material is present in spherical form.

11. A Process according to claim 8, wherein the adsorbent is subjected to an activation in a step d) after step c).

12. A Process according to claim 8, wherein the support material is binder free.

13. A Process according to claim 8, wherein the Si/Al ratio of the zeolite is in the range from 1.0 to 1.5, preferably from 1.15 to 1.25.

14. A Process according to claim 8, wherein the support material is, in step b), firstly subjected to barium ion exchange and then to magnesium ion exchange.

15. A Process according to claim 8, wherein the support material is, in step b), firstly subjected to magnesium ion exchange and then to barium ion exchange.

16. A Process according to claim 8, wherein the ion exchange is effected from a solution containing barium and magnesium ions.

17. The Use of an adsorbent according to any of claim 1 for separating para-xylene from an aromatic hydrocarbon mixture containing C8-aromatics.

18. The Use according to claim 17, wherein the separation is carried out in the gas phase.

19. The Use according to claim 17, wherein the separation is carried out in the liquid phase.
